(19) United States
(12) Patent Application Publication
(10) Pub. No.: US 2012/0121518 A1
(43) Pub. Date: May 17, 2012

(54) Multifunctional imaging and therapeutic nanoparticles

(76) Inventors: Zhihua Lu, Johns Creek, GA (US); Thomas Hirt, Rebstein (CH)

(21) Appl. No.: 13/373,499

(22) Filed: Nov. 16, 2011

Related U.S. Application Data
(60) Provisional application No. 61/456,993, filed on Nov. 16, 2010.

(51) Int. Cl. A61K 49/12; A61K 47/34; B32B 5/16; C08G 65/338; C08K 9/12; B82Y 25/00; B82Y 5/00; B82Y 15/00

(52) U.S. Cl. 424/9.323; 523/105; 514/772.1; 428/402; 977/773; 977/906; 977/930

(57) ABSTRACT

Multifunctional nanoparticles for imaging and therapeutic drug delivery made from amphiphilic block copolymers having a hydrophobic block and one or more hydrophilic blocks, and at least one internal and one terminal chelating agent units having a paramagnetic metal ion associated therewith.

### RELATED APPLICATION

The present application is related to and claims priority to U.S. Provisional Application Ser. No. 61/456,993 filed Nov. 16, 2010, the entire contents of which are incorporated herein by reference.

### STATEMENT REGARDING FEDERALLY SPONSORED RESEARCH OR DEVELOPMENT

This invention was made with government support under contract number HHSN261200900081C awarded by National Cancer Institute. The government has certain rights in the invention.

### BACKGROUND OF THE INVENTION

This invention relates generally to multifunctional nanoparticle drug delivery systems that can be detected using magnetic resonance imaging (MRI). More specifically, the present invention relates to polymeric amphiphilic nanoparticles that can be detected with MRI and that are suitable for delivery of active agents.

There is a growing interest in biological nanocarriers. Incorporation of an imaging agent into a nano-sized drug delivery system can allow for monitoring of drug delivery efficiency and real time analysis of any therapeutic response. One example of multifunctional nanotechnology is described in U.S. Pat. No. 7,459,145. This patent describes magnetic nanoparticles having nucleic acid or polypeptide probes coated on their surfaces. The nanoparticles bind to a target and generate an interaction observable with MRI or optical imaging. In order to incorporate additional function, these nanoparticles can further comprise a fluorescent or luminescent resonance energy transfer moiety or hydrophilic drugs.

Another example of multifunctional nanotechnology is described in United States Patent Application No. 2008/0019908. This disclosure teaches an amphiphilic polypeptide nanoparticle system which can be used as a nanocarrier for drug delivery and contrast agent delivery. The hydrophilic block is a hydrophilic polypeptide chain having sarcosine units and contains imaging contrast agent, and the hydrophobic block is a hydrophobic molecular chain comprising amino acid units or hydroxyl acid units. An issue with this approach is that peptide synthesis based on a defined platform is not cost effective.

The prior art describes multifunctional nanocarriers of imaging agents and drugs. However, new systems are desirable.

### SUMMARY OF THE INVENTION

The present invention describes multifunctional nanoparticles. The nanoparticles can deliver one or more active agents and are detectable via MRI. The nanoparticles are made from amphiphilic block copolymers having hydrophobic and hydrophilic blocks. The copolymers also include chelating units carrying MRI imaging agents. Each copolymer includes two or more chelating units, at least one of which is internal and one is terminal, each linked to a hydrophilic unit.

In a preferred embodiment the hydrophobic blocks are degradable polyesters. These can carry hydrophobic active agents. In a preferred embodiment, the MRI imaging agents are paramagnetic metal ions.

This invention further provides a method of forming multifunctional nanoparticles having imaging and drug delivery functions.

The term “nanoparticles” refers to micelles, nanospheres, nanocapsules, and nanoparticles having a size up to about 500 microns.

The term “block copolymers” refers to linear block copolymers as well as various other structures, such as graft and comb structures, containing both A and B segments.

### DETAILED DESCRIPTION OF THE INVENTION

The present invention describes multifunctional nanoparticles. The nanoparticles carry one or more active agents and are detectable via MRI. The nanoparticles are made from amphiphilic block copolymers having hydrophobic and hydrophilic blocks. The copolymers also include chelating units carrying MRI imaging agents. Each copolymer includes two or more chelating units, at least one of which is internal and one is terminal, each linked to a hydrophilic unit.

In preferred embodiments, the multifunctional nanoparticles are made from amphiphilic block copolymers having the structure B-x₁-(A₁-x₂)ₙ-A₂-x₃ or A₁-B-x₁-(A₂-x₂)ₙ-A₃-x₃ where B is a hydrophobic block, x₁ and x₂ are linking units or chelating units and x₃ is a chelating unit, A, A₁ and A₂ are hydrophilic blocks, n is from 0 to 10, and x₁ is a chelating unit if n is 0. At least one paramagnetic metal ion is chelated to each x which is a chelating unit.

Examples of hydrophobic blocks B are degradable polyesters such as polyhydrooxybutyrate, polyhydroxyvalerate, polycaprolactone, polyalkylcaprolactone, polylactic acid, polyglycolic acid, and copolymers thereof.

Examples of hydrophilic blocks A are polyethylene glycol, polyoxazoline, and copolymers thereof. Diamino oligoethylene glycols and amino polyethylene glycols can be prepared in good yields by using the reaction of sodium diformamide with oligoethylene glycol or polyethylene glycol dichlorides or ditosylates followed by hydrolysis using ethanolic hydrochloride as taught by Han et al. in Synthetic Communications, 21(1), January 1991, 79-84.

Examples of chelating units are ethylenediamine-N,N,N′,N′-tetraacetic acid (EDTA), N,N,N′,N″,N″-diethylenetriaminepentaacetic acid (DTPA), 1,4,7,10-tetraazacyclododecane-N,N′,N″,N″′-tetraacetic acid (DOTA), 1,4,7,10-tetraazacyclododecane-N,N′,N″-triacetic acid (DO3A), 1-oxa-4,7,10-triazacyclododecane-N,N′,N″-triacetic acid (OTTA), and trans(1,2)-cyclohexanodiethylenetriamine pentaacetic acid (CDTPA).

Examples of paramagnetic metal ions are Cr, V, Mn, Fe, Co, Ni, Cu, Ce, Pr, Nd, Pm, Sm, Eu, Gd, Tb, Dy, Ho, Er, Tm, and Yb. Especially preferred are Cr³⁺, Fe³⁺, Gd³⁺, and Dy³⁺.

The mean molecular weight of the A blocks is desirably in the range from about 500 to about 40,000, preferably in the range from about 1000 to 20,000. The B blocks desirably have a molecular weight of about 500 to 20,000, preferably between about 1000 and 10,000.

Preparation of the Amphiphilic Copolymers

Hydrophilic and hydrophobic groups can be connected through synthetic methods known in the art. For example, U.S. Pat. No. 6,916,488 describes ways to make amphiphilic copolymers. Coupling methods can be used to incorporate chelating units between the A and B groups and to attach a chelating unit to the terminal hydrophilic group. Preferably, a chelating unit is incorporated into the backbone by using its dianhydride as an agent to link it to adjoining hydrophilic or hydrophobic units. Alternatively, a non-chelating linking agent can be used between hydrophobic and hydrophilic blocks. Examples of linking agents are dianhydrides and diisocyanates.

Chelation of Metal Ions

Chelating a paramagnetic metal ion with a chelating unit on the amphiphilic copolymer can be done by a number of means known to those skilled in the art. Preferably, paramagnetic metal ion is combined with amphiphilic polymer in aqueous solution or alcohol/water mixture. Excess paramagnetic metal ion can be removed by dialysis or diafiltration.

Making Nanoparticles from the Amphiphilic Copolymers

The amphiphilic copolymers can be made into nanoparticles by a number of methods known to those skilled in the art. Self assembly techniques are preferred. In a typical procedure, the amphiphilic block copolymer is dissolved in a solvent such as ethanol at a concentration up to 50% w/v. The polymer solution is mixed with saline, with stirring. This procedure generally leads to a dispersion of copolymer nanoparticles of a rather broad size distribution. The size distribution can be controlled by methods known to those skilled in the art of preparing nanoparticles. In addition, the size distribution can be selected by passing the polydisperse particles through one or more filters having a defined pore size. The resulting particle dimensions are directly determined by the pore diameter of the filter membrane. In another procedure, for non-solvent formulation, the solvent is removed under reduced pressure and the resulting mixture of amphiphilic block copolymers is dispersed in saline, with stirring, to form nanoparticles as above.

Desirably, to facilitate entry into the cell, nanoparticles ranging from about 10 to 1000 nm in diameter, more preferably about 20 nm to about 500 nanometers in diameter, are used.

Method for Delivering a Hydrophobic Drug

The drugs may be encapsulated into the polymer by at least two different routes. In one method, the drug may be directly added to the copolymer during preparation of the copolymer. For example, the drug may be dissolved together with the polymer in ethanol.

The particles are suitable for delivery of a large number of therapeutic, diagnostic, or prophylactic agents (collectively referred to as “therapeutic agent” or “drug”). In one embodiment, the therapeutic agent is delivered to the target site via diffusion through the particle, and not by degradation of the particle. The polymer shell should be permeable to the agent in order to properly deliver the encapsulated drug.

Drugs can be proteins or peptides, polysaccharides, lipids, nucleic acid molecules, or synthetic organic molecules. Examples of hydrophobic compounds include some chemotherapeutic agents such as cyclosporine and taxol. These can be hormones, chemotherapeutics, antibiotics, antivirals, antifungals, vasoactive compounds, immunomodulatory compounds, vaccines, local anesthetics, antiangiogenic agents, antibodies, neurotransmitters, psychoactive drugs, drugs affecting reproductive organs, and antisense oligonucleotides. Diagnostic agents include gas, radiolabels, magnetic particles, radiopaque compounds, and other materials known to those skilled in the art.

Although described here primarily with reference to drugs, it should be understood that the particles can be used for delivery of a wide variety of agents, not just therapeutic or diagnostic agents. Examples include fragrances, dyes, photoactive compounds, reagents for chemical reactions, and other materials requiring a controlled delivery system.

The nanoparticles can be used as experimental, diagnostic, and therapeutic reagents. For therapeutic usage, the nanoparticles can be administered orally, by injection or by pulmonary, mucosal, or transdermal routes. The nanoparticles will usually be administered in combination with a pharmaceutically acceptable carrier. Pharmaceutical carriers are known to those skilled in the art. The appropriate carrier will typically be selected based on the mode of administration.

Dosing is dependent on severity and responsiveness of the condition to be treated, but will normally be one or more doses per day, with course of treatment lasting from several days to several months or until the attending physician determines no further benefit will be obtained. Persons of ordinary skill can determine optimum dosages, dosing methodologies, and repetition rates.

Targeting of Multifunctional Nanoparticles

The particles can be targeted to a particular site using targeting molecules bound to the surface, or extending from within to the surface, of the particles, where the molecules specifically or preferentially bind to a particular cell or tissue specific site. Examples of targeting molecules include carbohydrates, proteins, folic acid, peptides, peptoids, and antibodies. The list of useful ligands to facilitate binding to mucous type tissues include sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, and fucose. Antibodies may be directed to specific cell surface molecules or to antigens expressed when a cell type becomes diseased, for example, a cancer marker.

Targeting can be achieved, for example, by copolymerization of the amphiphilic copolymers with a small fraction of ligand-bearing comonomers, e.g. galactosyl-monomers. It is well known that such polymer-bound galactosyl-groups are recognized by the receptors at the surface of hepatocytes (Weigel, et al. J. Biol. Chem. 1979, 254, 10830). Such labeled nanoparticles will migrate to the target.

### EXAMPLES

The examples below serve to further illustrate the invention, to provide those of ordinary skill in the art with a complete disclosure and description of how the compounds, compositions, articles, devices, and/or methods claimed herein are made or could be made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

Materials and equipment: All reagents, solvents were of reagent grade and were purchased from commercial sources such as Polysciences, Fluka, Aldrich and Sigma.

General analysis: The polymers synthesized according to these examples can be chemically analyzed using structure-determining methods such as nuclear (proton and carbon-13) magnetic resonance spectroscopy, infrared spectroscopy and UV-visible spectroscopy. Polymer molecular weights can be determined using gel permeation chromatography. Aqueous solution properties such as micelle and nanoparticle formation can be determined using fluorescence spectroscopy, UV-visible spectroscopy, and laser light scattering instruments.

### Example 1

### PMCL-DTPA-PEG-OH, an Example of B-x-A-OH

Poly(4-methyl-ε-caprolactone) (PMCL) can be synthesized according to published literature, see Ren-Shen Lee and Chia-Bin Hung, Polymer 2007, 48 (9): 2605-2612. PMCL60-OH (2.0 g) is dried under vacuum at 60° C. for 12 h. DTPA dianhydride (0.357 g) and anhydrous dimethylformamide (50 ml, DMF) are added under nitrogen. The mixture is kept at 60° C. and stirred for 24 h. Polyethylene glycol diol or diamine (PEG45, MW 1450, 20 g) is dissolved in 30 ml of anhydrous toluene and transferred to the solution. After 24 h at 60° C., the solvent is removed under reduced pressure. The product is dispersed in water and filtered. The polymer is further purified by diafiltration through a 1 kDA molecular weight cutoff membrane in water/alcohol. After the solvent is removed under reduced pressure, the product is dried under vacuum at 60° C. for 12 h.

This product can be used to couple additional (x-A) units and/or a chelator unit can be attached to the end.

### Example 2

### PMCL-DTPA-PEG-DTPA, an Example of B-x-A-x

PMCL30-OH 1.90 g and polyethylene glycol (0.787 g, Mw 1450) are dried under vacuum at 60 C for 12 h. DTPA dianhydride (0.396 g) and 25 ml of anhydrous DMF are added under nitrogen. The mixture is kept at 60° C. and stirred for 24 h. After removal of solvent under reduced pressure, the product is dispersed in water for 12 h under magnetically stirring and filtered. Polymer is further purified by diafiltration through 1 kDa molecular weight cutoff membrane in water/alcohol. After the solvent is removed under reduced pressure, polymer is dried under vacuum at 60° C. for 12 h.

### Example 3

### PEG-PMCL-DTPA-PEG-DTPA-PEG-DTPA, an Example of A-B-x-(A-x)₂

In similar procedure as example 2, this polymer is made by using PEG45-PMCL15-OH 1.95 g, polyethylene glycol (Mn 400 g/mol, 0.40 g), DTPA dianhydride (0.55 g) and 25 ml of anhydrous DMF. PEG45-PMCL15-OH (1.95 g) and polyethylene glycol (0.40 g, Mw 400) are dried under vacuum at 60° C. for 12 h. DTPA dianhydride (0.55 g) and 25 ml of anhydrous DMF are added under nitrogen. The mixture is kept at 60° C. and stirred for 24 h. After removal of solvent under reduced pressure, the product is dispersed in water for 12 h while magnetically stirring and filtered. The polymer is further purified by diafiltration through 1 kDa molecular weight cutoff membrane in water/alcohol. After the solvent is removed under reduced pressure, the polymer is dried under vacuum at 60° C. for 12 h.

### Example 4

### PEG-PMCL-DTPA-PEG-DTPA-PEG-DTPA/3Gd-Gd³⁺-Containing Amphiphilic Polymer

GdCl₃ is added to a water/ethanol (1/1, v/v) solution of the copolymer of Example 3 (1.05 molar equivalents of Gd to DTPA). The mixture is stirred for 3 h and diafiltrated against a 1 kDa cutoff membrane. After the solvent is removed under reduced pressure, the polymer is dried under vacuum at 60° C. for 12 h.

### Example 5

### Formation of Hydrophobic Drug-Containing Amphiphilic Polymer Nanoparticles

Paclitaxel (1 mg) and PEG23-PMCL27-DTPA-PEG9-DTPA-PEG9-DTPA/3Gd (40 mg) are dissolved in 40 ul ethanol. The solution is diluted with 3 ml of 0.9% saline and vortexed for 15 seconds. The solution is extruded through a filter of 0.2 um pore size. Light scattering analysis shows that the nanoparticles have a size distribution of 50-200 nm.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A multifunctional nanoparticle formed from an amphiphilic block copolymer having the the structure B-x₁-(A₁-x₂)ₙ-A₂-x₃ or A₁-B-x₁-(A₂-x₂)ₙ-A₃-x₃ where B is a hydrophobic block, x₁ and x₂ are linking units or chelating units and x₃ is a chelating unit, A, A₁ and A₂ are hydrophilic blocks, n is from 0 to 10, x₁ is a chelating unit if n is 0, and at least one paramagnetic metal ion is chelated to each x which is a chelating unit.

2. The multifunctional nanoparticle of claim 1 further comprising a hydrophobic active agent.

3. The multifunctional nanoparticle of claim 1 wherein the hydrophobic block is a hydrophobic degradable polyester.

4. The multifunctional nanoparticle of claim 1 wherein the hydrophilic block is selected from the group consisting of polyethylene glycol or polyoxazoline.

5. The multifunctional nanoparticle of claim 1 wherein the hydrophobic block is selected from the group consisting of polyhydrooxybutyrate, polyhydroxyvalerate, polycaprolactone, polyalkylcaprolactone, polylactic acid, polyglycolic acid, and copolymers thereof.

6. The multifunctional nanoparticle of claim 1 wherein the linking unit is selected from dianhydride and diisocyanate.

7. The multifunctional nanoparticle of claim 1 wherein the chelating unit is selected from ethylenediamine-N,N,N′,N′-tetraacetic acid (EDTA), N,N,N′,N″,N″-diethylenetriaminepentaacetic acid (DTPA), 1,4,7,10-tetraazacyclododecane-N,N′,N″,N″′-tetraacetic acid (DOTA), 1,4,7,10-tetraazacyclododecane-N,N′,N″-triacetic acid (DO3A), 1-oxa-4,7,10-triazacyclododecane-N,N′,N″-triacetic acid (OTTA), and trans(1,2)-cyclohexanodiethylenetriamine pentaacetic acid (CDTPA).

8. The multifunctional nanoparticle of claim 1 wherein the paramagnetic metal ion is selected from Cr, V, Mn, Fe, Co, Ni, Cu, Ce, Pr, Nd, Pm, Sm, Eu, Gd, Tb, Dy, Ho, Er, Tm, and Yb.

9. The multifunctional nanoparticle of claim 1 having a particle size of 10 to 500 nm.

10. The multifunctional nanoparticle of claim 1 further comprising a targeting molecule.
